(19) United States
(12) Patent Application Publication
(10) Pub. No.: US 2008/0114386 A1
(43) Pub. Date: May 15, 2008

(54) METHOD OF PROVIDING CORNEAL TISSUE AND METHOD OF DETERMINING THE BIOBURDEN OF LABORATORY PROVIDING SAME

(76) Inventors: Bernardino Iliakis, Seattle, WA (US); Thomas D. Miller, Everett, WA (US); Monty M. Montoya, Bellevue, WA (US)

Correspondence Address: DAVIS WRIGHT TREMAINE, LLP/Seattle, 1201 Third Avenue, Suite 2200, SEATTLE, WA, 98101-3045, US

(21) Appl. No.: 11/558,333

(22) Filed: Nov. 9, 2006

(51) Int. Cl. A61F 9/00; A61B 18/18; G06Q 50/00

(52) U.S. Cl. 606/166; 606/4; 705/2

(57) ABSTRACT

A method of preparing custom corneal tissue in response to a request for the custom corneal tissue that includes specifications specifying the size and shape of the custom corneal tissue. After the request is received, donor eye tissue is selected from a plurality of donor eye tissues. The plurality of donor eye tissues may be stored by an eye bank facility. Next, preparations are made to cut the selected donor eye tissue in accordance with the specifications included in the request. A set of parameters is determined from the specifications and used to design a cutting path. The selected donor eye tissue is cut along the cutting path to produce the custom corneal tissue. The custom corneal tissue is separated from the selected donor eye tissue, packaged, and provided to the requestor.

### BACKGROUND OF THE INVENTION

1. Field of the Invention

The present invention is directed generally to methods of extracting, dissecting, or customizing corneal tissue from donor eye tissue for transplantation into a patient, methods of providing corneal tissue with a custom boundary defined by a set of specifications developed for a specific corneal transplant patient, and methods of determining an acceptable bioburden of a laboratory processing human-tissue based products, such as corneal tissue.

2. Description of the Related Art

Referring to FIG. 1, a partial side cross-section of the front most portion of the human eye is provided. A portion of corneal tissue 12 has been cut from the cornea 10 in accordance with a prior art method of extracting corneal issue for transplant into a patient.

The cornea 10 is a transparent lens-shaped exterior structure of the eye that allows light to pass into the pupil (not shown). An anterior chamber 50 filled with clear aqueous humor is located behind the cornea 10 and between the cornea 10 and the pupil. The anterior chamber 50 is separate from and anterior to the posterior chamber 55. A portion of the outer boundary of the posterior chamber 55 is defined by the sclera 60, which is an opaque (usually white), fibrous, protective outer layer of the eye. The sclera 60 surrounds the cornea 10 and is integrally connected with the perimeter of the cornea.

The cornea 10 and lens (not shown) refract light and focus it on the retina (not shown). Muscles within the eye flex the lens to adjust the refraction of light passing through the lens. While the cornea 10 refracts light more than the lens, the eye does not include any structures for flexing or otherwise adjusting the refraction of light passing through the cornea 10. In humans, the refractive power of the cornea 10 may be approximately 43 diopters, which is about 75% of the total refractive power of the eye.

The cornea 10 does not have blood vessels. Instead, nutrients diffuse into the cornea 10 from the tear fluid disposed along the outside surface and aqueous humor disposed along the inside surface of the cornea 10. The cornea 10 may also receive neurotrophins from nerve fibres that innervate the cornea 10. In humans, the cornea 10 has a diameter of about 11.5 mm and a thickness of about 0.5 mm to about 0.6 mm near the center and about 0.6 mm to about 0.8 mm near the periphery of the cornea 10.

Referring to FIG. 1, the cornea 10 has three layers of tissue: corneal epithelium 20, stroma 30, and corneal endothelium 40. The corneal epithelium 20 covers the front of the cornea 10 and consists of several layers of cells. The stroma 30 (also known in the art as the substantia propria) is a fibrous structure that includes about sixty flattened lamellae. These lamellae are constructed from bundles of modified connective tissue. The fibers present in the modified connective tissue near the periphery of the stroma 30 are directly continuous with the fibers of the sclera 60. The fibers of a single lamella of the stroma 30 are generally parallel with one another and generally orthogonal to the fibers of adjacent lamellae. The fibers of one lamella may pass into another.

The corneal endothelium 40 is a monolayer of specialized, flattened, mitochondria-rich cells that lines the posterior surface of the cornea 10 and faces the anterior chamber 50 behind the cornea 10. The corneal endothelium 40 governs fluid and solute transport across the posterior surface of the cornea 10 and actively maintains the transparency of the cornea 10. Damage or disease of the corneal endothelium 40 is a predominant reason a patient may need a cornea transplant (also referred to in the art as a corneal graft or penetrating keratoplasty).

Donor corneal tissue for cornea transplants is collected, stored, and made available to eye surgeons and researchers by eye bank facilities. Eye bank facilities are generally located off-site and in a separate facility from the surgeon or researcher requesting the donor eye tissue. In some cases, the eye bank facility may ship the donor eye tissue to the surgeon or researcher.

An eye bank facility typically stores a piece of donor eye tissue referred to as a corneo-scleral button that includes both the cornea 10 and part of the white sclera 60 in a container filled with a preservation medium such as Optisol-GS (“Optisol”) manufactured by BAUSCH & LOMB of Irvine, Calif. Hereafter, the corneo-scleral button that includes both the cornea 10 and part of the white sclera 60 will be referred to as the “donor eye tissue.” After the donor eye tissue has been approved for transplant, the eye bank facility simply ships the container containing the donor eye tissue to a surgeon who performs the dissection of the donor eye tissue and extracts the portion of the cornea needed for the transplant surgery.

The prior art method of extracting the corneal tissue 12 from donor eye tissue illustrated in FIG. 1 includes cutting through all three layers of the cornea 10 to obtain a detached full-thickness piece of lens-shaped corneal tissue 12. A trephine (not shown), which is a surgical instrument with a cylindrical blade, may be used to manually extract a circular piece (or button) of corneal tissue. The edge profile 70 obtained using this technique is generally vertical and linear. The trephine is also used to remove corneal tissue from the patient's eye in the same manner. The trephine is capable of cutting a circular piece of tissue of a predetermined diameter and cannot be used to cut pieces having alternate diameters. Therefore, this technique is not amenable to customization. Further, control of the depth of the cut is limited by the dexterity, patience, and/or skill of the surgeon cutting the donor eye tissue.

The generally vertical and linear edge profile 70 of the donor corneal tissue is placed adjacent to a similar generally vertical and linear edge profile cut into the patient's cornea along the perimeter of the corneal tissue removed. Because the adjacent edge profiles are generally linear, the donor corneal tissue may slide relative to the edge of the remaining portion of the patient's cornea. Misalignment of the top surface of the donor corneal tissue and the top surface of the remaining portion of the patient's cornea may impair the patient's vision and result in astigmatism.

An alternate technique of dissecting or extracting corneal tissue from donor eye tissue uses a microkeratome, which is a surgical instrument having an oscillating blade, to cut a corneal flap or lens-shaped piece of corneal tissue from the eye. This slice may extend through only the corneal epithelium 20 and the majority of the stroma 30. The flap may be peeled back or the lens-shaped piece of corneal tissue removed. Then, a trephine may be used to extract any remaining stroma 30 and corneal endothelium 40. Next, the microkeratome may be used to cut a flap in patient's cornea. The thickness of the flap may extend though the majority of the stroma 30. The flap may be peeled back and the trephine used to extract the remaining stroma 30 and corneal endothelium 40 uncovered by peeling back the flap cut into the cornea 10. Then, the replacement tissue cut from the donor eye tissue may be inserted into the patient's eye under the flap. Finally, the flap is folded back to cover the transplanted replacement tissue.

More recently, femtosecond laser apparatuses of the type used to perform laser-assisted in situ keratomileusis (“LASIK”) surgery have been used to perform cornea transplants. One example of a commercially available femtosecond laser apparatus is a IntraLase™ FS laser manufactured by Intralase Corp. A laser of a femtosecond laser apparatus may use infrared light to precisely cut tissue by a process known as photodisruption. As used herein, the term “cut” includes an incision or cleavage plain such as the type created using the process of photodisruption.

Photodisruption involves the delivery of a large quantity of energy to a small focal spot over a brief duration. The energy causes a highly localized temperature increase that transforms the tissue within the small focal spot into plasma. However, the procedure is considered non-thermal because the heat quickly dissipates outwardly into surrounding tissue. Both the pressure and temperature destroys the tissue and causes the formation of a cavitation bubble containing carbon dioxide and water vapor. The cavitation bubble separates the lamellae of the cornea 10. The carbon dioxide and water vapor are absorbed by the endothelial pump mechanism of the cornea 10 leaving an incision or cleavage plane between the lamellae of the cornea 10. Thousands of cavitation bubbles may be created in raster or spiral patterns to create larger incisions or cleavage planes separating portions of the lamellae of the cornea 10. These incisions or cleavage planes may be oriented in any direction and created at any depth in the tissue of the cornea 10.

A software program may be used to direct and focus the beam of the laser onto a spot having an area of about 2 μm to about 3 μm. The beam may pass harmlessly through an outer portion of the cornea 10 until the beam reaches a focal point within the cornea 10. At the focal point, photodisruption occurs and a cavitation bubble is formed. The software program may direct the laser to create the thousands of cavitation bubbles required to create a larger incision or cleavage plane.

Referring to FIGS. 2A-2E, the femtosecond laser apparatus may be used to obtain more sophisticated edge profiles than the generally vertical and linear edge profile 70 depicted in FIG. 1. Illustrative examples of more sophisticated edge profiles include the edge profiles 70 depicted in FIGS. 2A-2E. Because the edge profiles 70 depicted in FIGS. 2A-2E are not generally linear and include surfaces that extend horizontally, the donor corneal tissue 12 may be less inclined to slide vertically relative to the edge of the remaining portion of the patient's cornea. Further, the edge profiles 70 of FIGS. 2A and 2C-2E include a horizontally extending surface 14 that abuts the underside of an overhang portion 16 of the patient's remaining cornea. The overhanging portion 16 may help maintain the donor corneal tissue 12 in the proper vertical position. Maintaining the donor corneal tissue 12 in the proper vertical position may help reduce astigmatism caused by vertical misalignment of the donor corneal tissue 12 relative to the remaining portion of the patient's cornea. The femtosecond laser apparatus may also be used to cut a customized piece of tissue from a patient's cornea and/or donor eye tissue. Further, the laser of the femtosecond laser apparatus may be used to cut an edge profile into the patient's cornea and a corresponding edge profile in the donor corneal tissue.

While the femtosecond laser apparatus may be used to remove both a portion of the patient's cornea and the corneal tissue from the donor eye tissue, the logistics of the operating room and the physical space requirements of the femtosecond laser apparatus make cutting both the patient's corneal tissue and the donor eye tissue in the operating room difficult. Consequently, the patient's cornea is typically cut by the femtosecond laser apparatus in a clinic before the patient enters the operating room. After the patient is taken to the operating room, the surgeon may use an instrument, such as a LASIK spatula, to separate the corneal tissues along the incisions and remove the patient's cornea. Similarly, the corneal tissue may be extracted from the donor eye tissue in the clinic outside the operating room and before the transplant surgery. Cutting the donor eye tissue in the clinic and transporting the tissue to the operating room takes time and adds logistical complexity to the cornea transplant operation.

Therefore, a need exists for methods related to preparing donor corneal tissue for transplantation. Further, a need exists for methods of preparing donor corneal tissue customized for a specific patient. A need also exists for methods of providing corneal tissue in response to a request including specifications describing the corneal tissue to be transplanted into the patient.

As is appreciated by those of ordinary skill in the art, before an organization, such as an eye bank facility, can process human tissue in a laboratory for transplantation, the organization must ensure its laboratory is not contaminated with bio-contamination, such as bacteria and fungi. For example, Current Good Tissue Practice for Manufacturers of Human Cellular and Tissue-Based Products under 21 C.F.R. § 1271.195 requires organizations that process human tissue-based products, such as donor eye tissue, to identify any environmental conditions that require monitoring and control. In particular, such organizations must monitor and control the environment in any laboratory where human tissue-based products are processed to prevent contamination of the tissue and/or cross-contamination between two or more separate tissues. As a result, a need exists for methods of determining the bioburden of a laboratory (i.e., the number of microorganisms with which the laboratory is contaminated) before, during, and/or after the laboratory processes human tissue-based products, such as donor eye tissue.

### BRIEF DESCRIPTION OF THE SEVERAL VIEWS OF THE DRAWING(S)

FIG. 1 is a partial side cross-sectional view of a front portion of an eye wherein a portion of the cornea has been removed in accordance with a prior art method of removing corneal tissue from donor eye tissue.

FIG. 2A is a side partial cross-sectional view of a cornea from which a portion of corneal tissue has been removed in accordance with a prior art method of removing corneal tissue from donor eye tissue.

FIG. 2B is a side partial cross-sectional view of a cornea from which a portion of corneal tissue has been removed in accordance with a prior art method of removing corneal tissue from donor eye tissue.

FIG. 2C is a side partial cross-sectional view of a cornea from which a portion of corneal tissue has been removed in accordance with a prior art method of removing corneal tissue from donor eye tissue.

FIG. 2D is a side partial cross-sectional view of a cornea from which a portion of corneal tissue has been removed in accordance with a prior art method of removing corneal tissue from donor eye tissue.

FIG. 2E is a side partial cross-sectional view of a cornea from which a portion of corneal tissue has been removed in accordance with a prior art method of removing corneal tissue from donor eye tissue.

FIG. 2F is a side partial cross-sectional view of a cornea from which a portion of corneal tissue has been removed in accordance with a prior art method of removing corneal tissue from donor eye tissue.

FIG. 3 is a block diagram illustrating an embodiment of the method of the present invention.

FIG. 4 is a block diagram illustrating an embodiment of a method in accordance with a block 1200 of the method of FIG. 3.

FIG. 5 is an exploded perspective view of an artificial anterior chamber assembly for use with the method of FIG. 3.

FIG. 6A is an elevational view of an exemplary laboratory for use with the present invention.

FIG. 6B is an enlarged fragmentary side view of a portion of the exemplary laboratory of FIG. 6A configured to perform the method of FIG. 3

FIG. 6C is an enlarged fragmentary cross-sectional view of the laboratory of FIG. 6B illustrating the interior of the artificial anterior chamber assembly of FIG. 5.

FIG. 7 is a block diagram illustrating an embodiment of a method in accordance with a block 1300 of the method of FIG. 3.

FIG. 8 is a block diagram illustrating an embodiment of a method for determining the bioburden of the laboratory of FIGS. 6A-6C before, during, and/or after performing the method of FIG. 3.

FIG. 9 is an elevational view of the exemplary laboratory of FIG. 6A including exemplary locations for sample collection in accordance with the method of FIG. 8.

### DETAILED DESCRIPTION OF THE INVENTION

Aspects of the present invention are directed toward a method of extracting a corneal tissue from donor eye tissue for transplantation into an eye of a patient. The transplantation procedures may include endothelial keratoplasty, anterior lamellar keratoplasty, and penetrating keratoplasty.

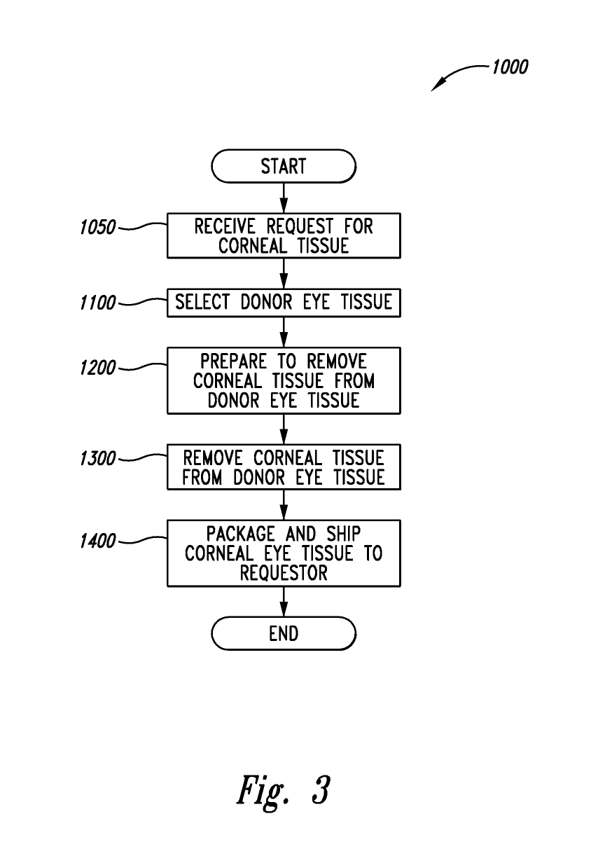

With reference to FIG. 3, a block diagram illustrating one embodiment of a method 1000 of the present invention will be described. The method 1000 may be performed by an organization that stores or otherwise possesses a plurality of donor eye tissues, such as an eye bank facility. As is appreciated by those of ordinary skill in the art, portions of the method 1000 may be performed in a laboratory.

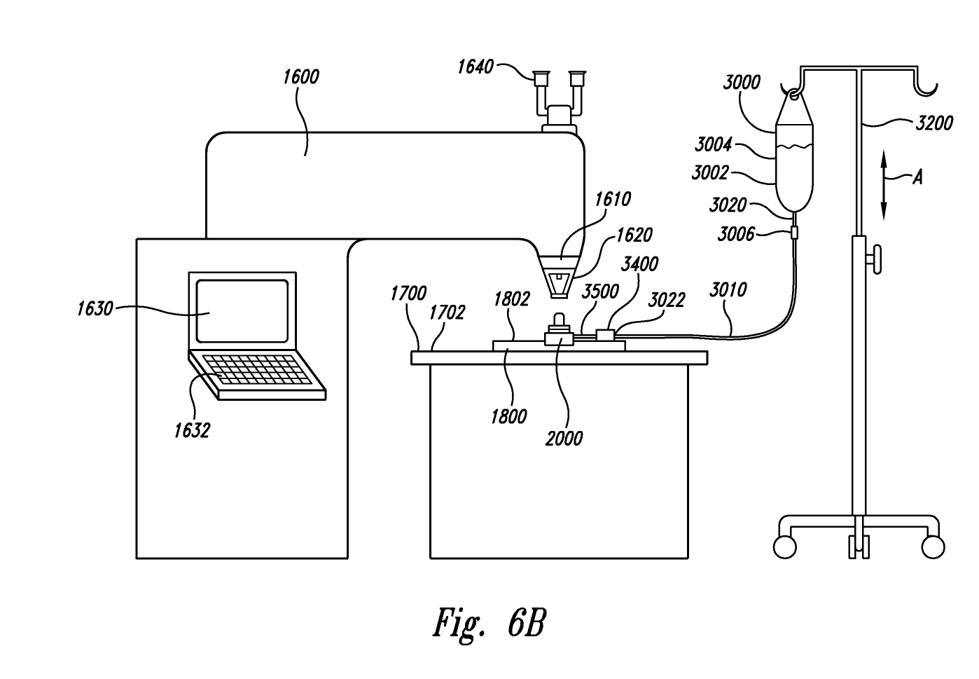

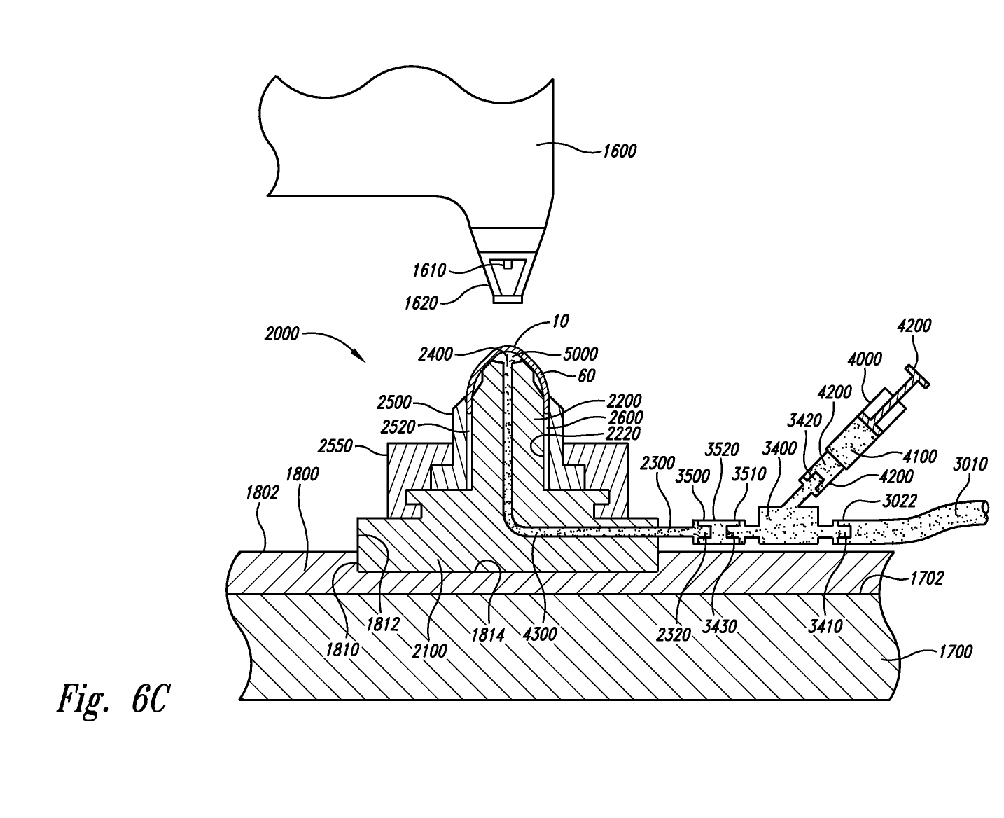

Referring to FIG. 6A, an exemplary embodiment of a laboratory in which the method 1000 may be performed is provided. The laboratory may include a laser apparatus 1600 located adjacent to a table 1700. Suitable laser apparatuses for use with the method 1000 include femtosecond laser apparatuses of the type used to perform LASIK surgery. The laboratory may also include fixtures and equipment of the type typically found in a laboratory such as a sink, preparation area, disposal, air conditioning unit (“A/C”), additional table(s), and power supply.

Referring to FIG. 6B, a portion of the exemplary embodiment of the laboratory of FIG. 6A configured to perform the method 1000 is provided. The table 1700 may include a substantially planar work surface 1702. In some embodiments, the laser apparatus 1600 may include a head 1610 from which the laser beam emanates configured to removably receive a sterile patient or tissue interface 1620. A rigid pad 1800 may be disposed on the work surface 1702 of the table 1700. A portion of the sterile instruments used to perform portions of the method 1000 may be placed on the work surface 1702 and or rigid pad 1800. Referring to FIG. 6C, the sterile instruments may include a sterile syringe 4000, syringe filter 4200, syringe needle (not shown), an artificial anterior chamber assembly 2000 (“AAC”), first tube 3010, second tube 3500, stopcock assembly 3400, and pen (not shown).

The instruments used to perform the method 1000 may include the AAC 2000. A suitable disposable AAC for use with the present invention includes a Barron Artificial Anterior Chamber model number K20-2125 which may be purchased from Katena Products, Inc. of 4 Stewart Court, Denville, N.J. 07834. A suitable reusable AAC for use with the present invention includes an Artificial Chamber manufactured by Moria, Inc. While an exemplary AAC is described herein and illustrated in the drawings, it is apparent to those of ordinary skill that alternate embodiments of AAC 2000 are known in the art and are within the scope of the present invention.

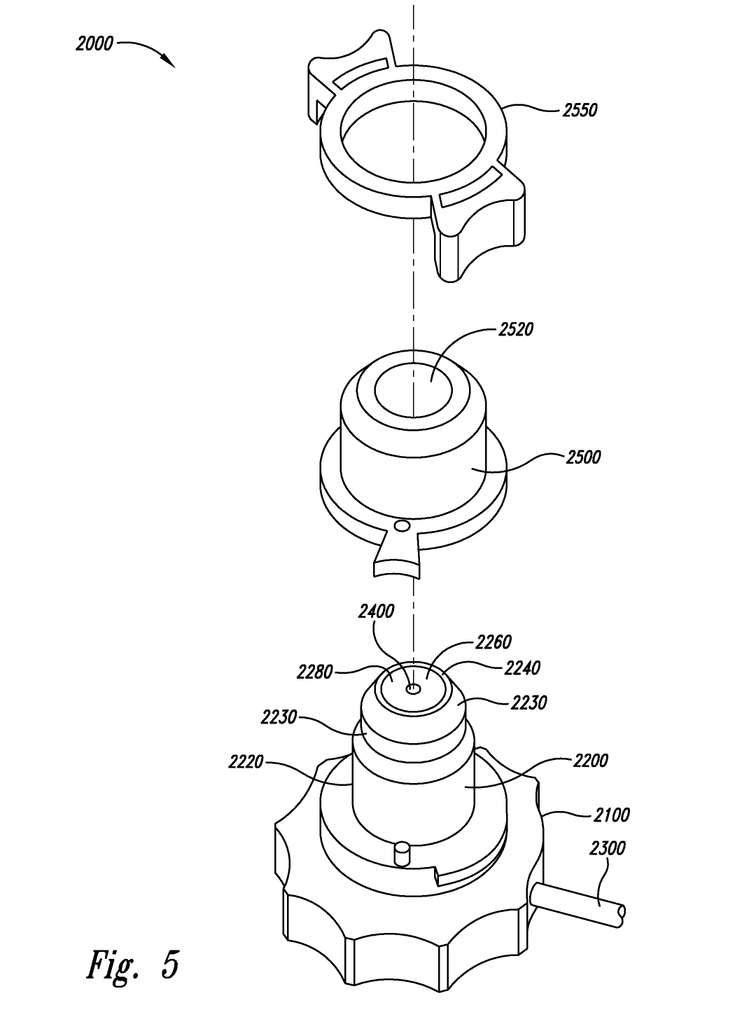

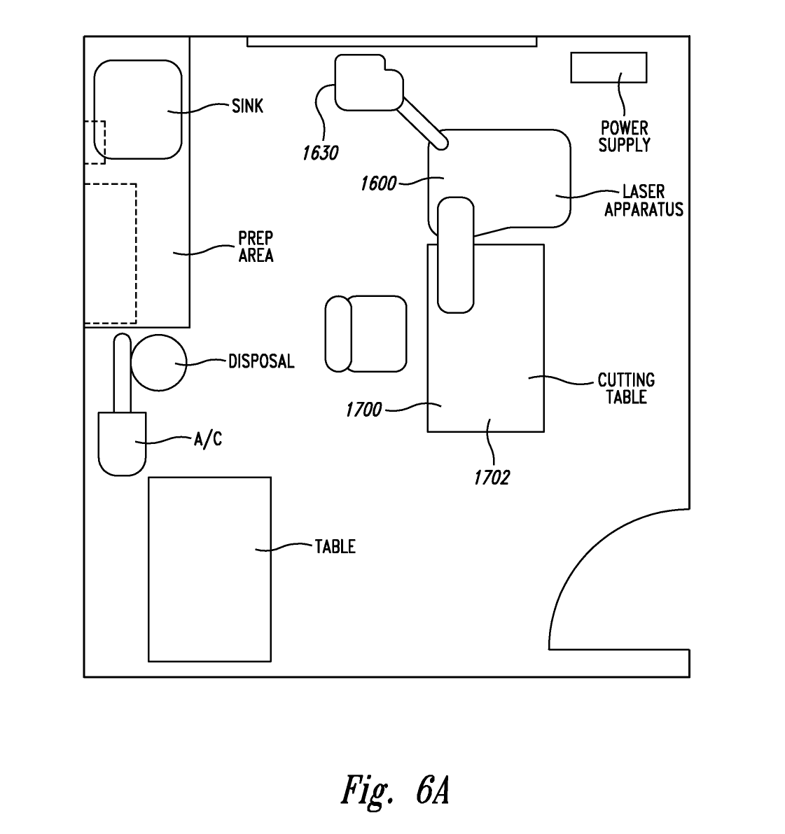

Referring to FIGS. 5 and 6C, the AAC 2000 may include a generally circular base 2100 with a generally cylindrical tissue pedestal 2200 that extends upwardly from the base 2100. One or more conduits or AAC tubes 2300 may extend from the exterior of the base, through a portion of the interior of the base, and up the inside of the tissue pedestal 2200. The tissue pedestal 2200 may include an exit aperture 2400 in fluid communication with the interior of each AAC tube 2300 to allow fluid inside the AAC tube 2300 to exit the AAC tube 2300.

Optionally, each AAC tube 2300 may include a connector 2320 (see FIG. 6C) to which a fluid source 3000 (see FIG. 6B) such as a bag, bottle, tank, reservoir, and the like may be connected. The fluid source 3000 houses a fluid 3002 such as normal saline, balanced salt solution, and the like. Each of the AAC tube(s) 2300 may include a valve (not shown) for limiting or preventing the flow of fluid into or from the AAC tube(s) 2300.

The tissue pedestal 2200 may include a generally vertically extending side surface 2220 intersecting a generally horizontal top surface 2240. The tissue pedestal 2200 may include a chamfered or relieved portion 2230 along the perimeter of the top surface 2240 near its intersection with the side surface 2220. A depression 2260 may be formed in a portion of the top surface 2240. The depression 2260 may define a cavity 2280 that has the general shape of a spherical cap. The exit aperture(s) 2400 may be formed in the top surface 2240. In one embodiment, the exit aperture(s) 2400 are formed in the depression 2260.

The AAC 2000 may include a tissue retaining ring or collar 2500 sized and shaped to removably receive the tissue pedestal 2200, as may best be viewed in FIG. 6C. The collar 2500 includes an inside surface 2520. A portion of the inside surface 2520 is adjacent to a portion of the side surface 2220 of the tissue pedestal 2200 when the tissue pedestal 2200 fully is received within the collar 2500. A gap 2600 is defined between the inside the portion of the inside surface 2520 of the collar 2500 that is adjacent to the portion of the side surface 2220 of the tissue pedestal 2200. Optionally, the AAC 2000 may include a locking member 2550 that secures the collar 2500 to the base 2100. Alternatively, the collar 2500 may include a lock (not shown) configured to secure the collar 2500 to the base 2100.

The fluid source 3000 may be coupled to the AAC tube(s) 2300 to introduce the fluid 3002 housed inside the fluid source 3000 into the interior of the AAC tube(s) 2300. The fluid 3002 inside the AAC tube(s) 2300 may exit the AAC tube(s) 2300 via the exit aperture(s) 2400.

Returning to FIG. 3, as with any corneal transplant procedure, a first block 1050 of method 1000 may start with a request for donor eye tissue. The request originates from a requestor, which may include an eye surgeon, researcher, surgery scheduler, and others who use donor eye tissue for any purpose. Instead of requesting the entire piece of donor eye tissue, the requestor requests only the cornea 10 or a portion thereof. The recipient of the request, such as the eye bank facility, may extract the requested tissue from the sclera 60 and/or cornea 10. Because the entire cornea 10 or a portion thereof may be requested, the term “corneal tissue” will be used hereafter to refer to the portion of the donor eye tissue requested by the requestor and subsequently prepared and provided by the recipient of the request.

The request for the corneal tissue may include specifications related to the characteristics of the corneal tissue such as thickness, size, shape, edge profile, and the like. The specifications may define a boundary of the corneal tissue. In an alternate embodiment, the specifications define the incisions to be cut along a portion of the boundary of the corneal tissue. In one embodiment, the specifications define the edge profile of the corneal tissue. The specifications may be used to program a laser apparatus 1600 (see FIGS. 6B and 6C) to cut the corneal tissue from the donor eye tissue.

After the request for corneal tissue is received, in a block 1100, the recipient selects a piece of donor eye tissue (from the plurality of donor eye tissues it possesses) from which to obtain the requested corneal tissue. The section process may include inspecting the donor eye tissue. The process of inspecting donor eye tissue to determine its suitability for dissection and transplantation into the patient is well known in the art.

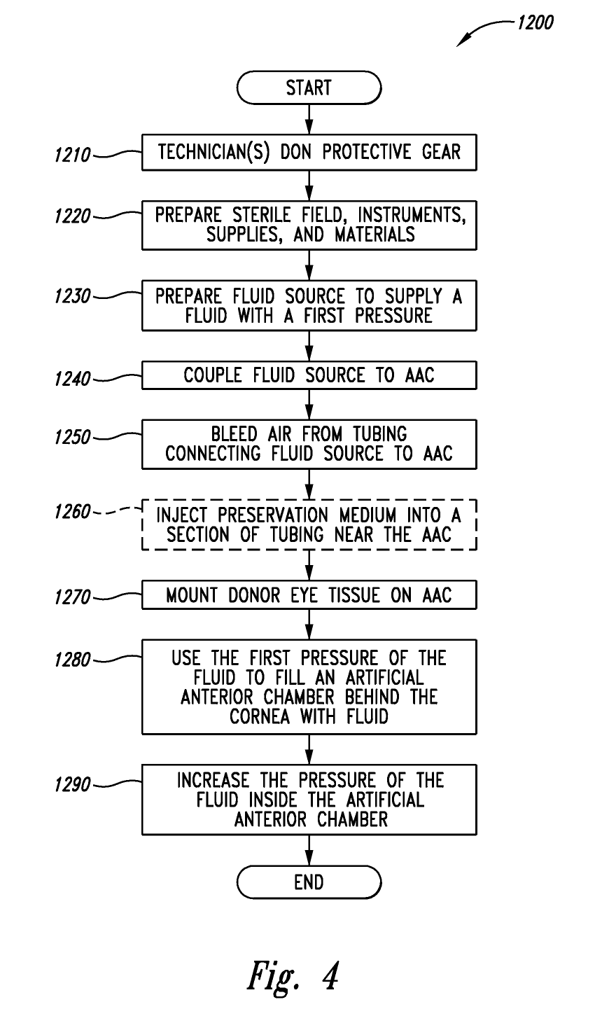

Then, in a block 1200, the recipient prepares to remove the corneal tissue from the donor eye tissue. FIG. 4 is a block diagram illustrating one embodiment of the method of the block 1200. Before the donor eye tissue can be handled by one or more technician(s), in a block 1210, the technician(s) may don protective gear such as sterile latex gloves, a mask with a face shield, a cap to cover his/her hair, a moisture impermeable gown to protect the technician(s) from potential exposure to infectious disease, and the like. The protective gear may be donned in the laboratory. The protective gear worn by the technician(s) may be determined by regulatory authorities such as the FDA and/or the Eye Bank Association of America and the present invention is not limited with respect to the protective gear worn by the technician(s).

In a next block 1220, the technician(s) prepare an aseptic environment that includes a sterile field and sterile instruments. As is appreciated by those of ordinary skill in the art, it may be beneficial to designate one or more technicians to work within the sterile field and/or handle sterile instruments and one or more technicians to work outside the sterile field and/or with non-sterile tools, instruments, and materials within the laboratory. Technicians who work outside the sterile field and/or with non-sterile tools, instruments, and materials may be referred to as circulators.

The sterile field may be established by wiping the work surface 1702 of the table 1700 with a towel (not shown) including a cleaning solution. Any suitable cleaning solution known in the art for sterilizing work surfaces and instruments may be used including CaviCide. In some embodiments, a sterile drape (not shown) is disposed on the work surface 1702 to provide a sterile area in which to cut the donor eye tissue. Methods of using the sterile drape to provide the sterile area are well known in the art. The sterile field may also include the rigid pad 1800 disposed on the work surface 1702 of the table 1700. Preparing the sterile field may including wiping a top surface 1802 of the rigid pad 1800 with the towel including the cleaning solution. In some embodiments, the sterile drape is disposed on top of top surface 1802 of the rigid pad 1800.

A portion of the instruments may be placed on the sterile field. Any sterile instruments, materials, and/or supplies required in subsequent steps and disposed within packaging may be inspected and opened onto the sterile field. Referring to FIG. 6C, the sterile instruments may include the sterile syringe 4000, syringe filter 4200, syringe needle (not shown), first tube 3010, second tube 3500, stopcock assembly 3400, pen (not shown), and tissue interface 1620.

The AAC 2000 may be purchased sterile and housed within packaging. The packaging may be opened and the AAC 2000 placed on the sterile field. Alternatively, the AAC 2000 may be cleaned using any method known in the art for sterilizing the AAC 2000 and placed on the sterile field.

Any portion of the laser apparatus 1600 that physically contacts the donor eye tissue may also be sterilized. In some embodiments, the head 1610 of the laser apparatus 1600 from which the laser beam emanates is configured to removably receive the sterile patient or tissue interface 1620. While in such embodiments the head 1610 does not physically contact the donor eye tissue, it may be desirable to sterilize the head 1610 by wiping it with the towel including the cleaning solution.

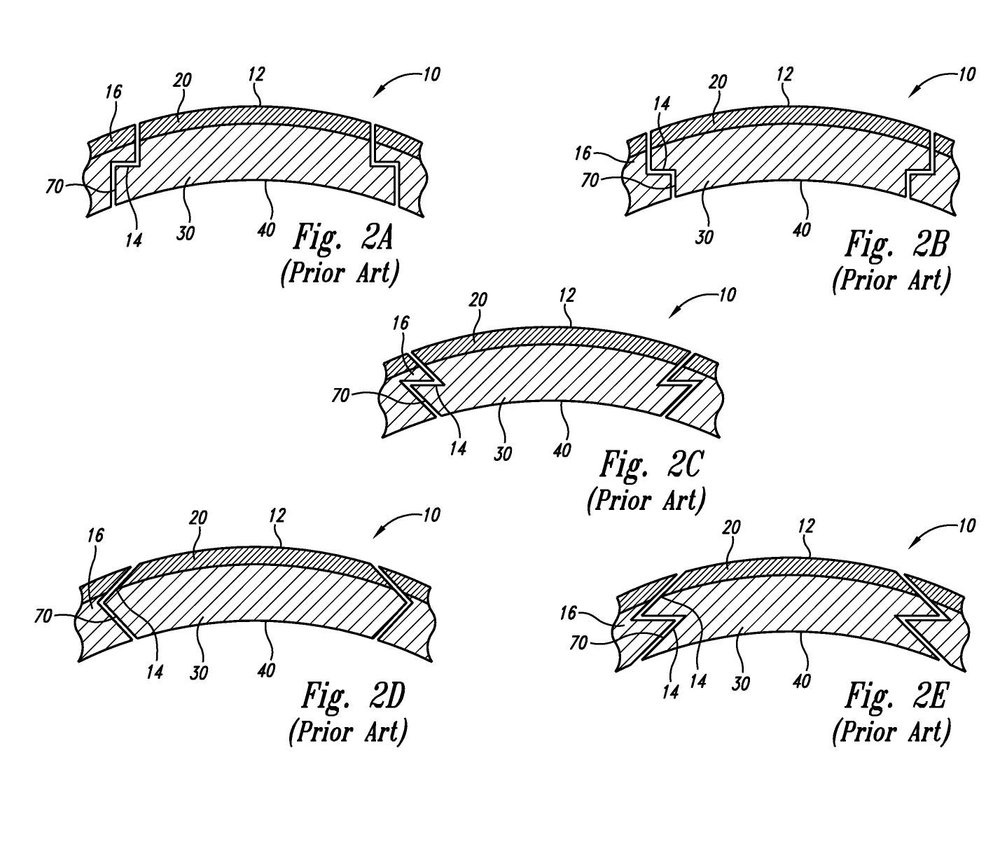

Referring back to FIG. 4, in a block 1230, the fluid source 3000 is prepared and configured to supply the fluid 3002 with a first pressure into a first tube 3010 (see FIG. 2B). In one embodiment, the fluid source 3000 may include a pre-filled, sterile container 3004 such as a glass bottle, plastic bottle, or plastic bag of the fluid 3002. The fluid source 3000 may include any standard IV infusion system known in the art for providing a steady stream of the fluid 3002 via a tube such as an IV line. A standard IV kit (not shown) well known in the art may be coupled to the container 3004. The IV kit may include a first tube 3010 such as IV line. One end 3020 of the first tube 3010 may be coupled to the fluid source 3000 to allow the fluid 3002 to flow from the container 3004 into the first tube 3010 using any manner known in the art.

The IV kit may include a drip chamber 3006. The container 3004 may be coupled to a drip chamber 3006 that allows the fluid 3002 to flow one drop at a time from the sterile container into the drip chamber 3006. The first tube 3010 may be coupled to a bottom port (not shown) of the drip chamber 3006 to allow the fluid 3002 from the fluid source 3000 residing in the drip chamber 3006 to flow into the first tube 3010. One of the circulators may couple the drip chamber 3006 to the fluid source 3000 and the first tube 3010 to the drip chamber 3006.

The IV kit may include an IV line valve (not shown). The IV line valve may be installed in the first tube 3010 to selectively limit and/or prevent the fluid 3002 from flowing therethrough.

As depicted in FIG. 6B, in one embodiment, the container 3004 includes a standard IV bag containing the fluid 3002. The container 3004 may be hung from a standard IV pole, hanger, or stand 3200 that permits vertical height adjustments in the directions indicated by double-ended arrow “A.” The container 3004 may be hung in a first vertical position by adjusting the IV stand 3200 to a first height and hanging the container 3004 therefrom.

The pressure of the fluid 3002 supplied the container 3004 may be determined by the height of the container 3004 on the IV stand 3200. Hydrostatic pressure is caused by the weight of a fluid and is the product of the height of the column of fluid, density of the fluid, and acceleration of gravity. Raising the container 3004 may increase the height of the column of fluid in a portion of the first tube 3010 that is above the work surface 1702. The taller column may exert more hydrostatic pressure at the lower end 3022 of the first tube 3010.

Referring back to FIG. 4, in a block 1240, the fluid source 3000 is coupled to one of the AAC tubes 2300 of the AAC 2000 in any manner known in the art including using standard IV and stopcock kits. Referring to FIGS. 6B and 6C, the stopcock kit may include a standard stopcock assembly 3400 having two inlet ports 3410 and 3420 and a single outlet port 3430. A first stopcock (not shown) may be disposed between one of the inlet ports 3410 and the outlet port 3430 and a second stopcock (not shown) may be disposed between the other inlet port 3420 and the outlet port 3430. The end 3022 of the first tube 3010 may be coupled to the inlet port 3410 coupled to the first stopcock. The first stopcock may be used to limit the flow of fluid 3002 from the fluid source 3000 through the first stopcock.

Before the first tube 3010 is coupled to the inlet port 3410, any air in the air the first tube 3010 may be bled therefrom by allowing the fluid 3002 from the fluid source 3000 to flow into the first tube segment 3010 and out the end 3022 of the first tube 3010 into a section of sterile gauze (not shown). Opening the IV line valve may allow the fluid 3002 to flow into the first tube segment 3010. After the air is bled from the first tube 3010, the IV line valve may be closed.

One end 3510 of a second tube 3500 may be connected to the outlet port 3430 of the stopcock assembly 3400. The other end 3520 of the second tube 3500 may be coupled to one of the AAC tubes 2300 of the AAC 2000. In this manner, the second tube 3500 is disposed between the outlet 3430 of the stopcock assembly 3400 and one of the AAC tubes 2300 of the AAC 2000.

Referring back to FIG. 4, in a next block 1250, air is bled from the tubing connecting the fluid source 3000 to the AAC 2000. Initially, both the first and second stopcocks are placed in closed positions preventing the flow of fluid therethrough. To bleed air from the tubing connecting the fluid source 3000 to the AAC 2000, the first stopcock is placed in an open position to allow the fluid 3002 from the fluid source 3000 to flow into the first tube segment 3010, through the first stopcock, into the second tube 3500, into the AAC tube 2300 of the AAC 2000, and out the exit aperture 2400. The fluid 3002 may push the column of air contained in the tubing downstream from the fluid 3002 out the exit aperture 2400 thereby bleeding the air from the tubing disposed between the fluid source 3000 and the exit aperture 2400. Then, the first stopcock may be placed in a closed position to prevent the fluid 3002 from flowing from the first tube segment 3010 through the first stopcock and into the second tube 3500.

Optionally, in a next block 1260, a preservation medium may be injected into the second tube 3500 connecting the outlet 3430 stopcock assembly 3400 to one of the AAC tubes 2300 of the AAC 2000. Referring to FIG. 6C, a sterile syringe 4000 may be filled with about 5 cc to about 6 cc of preservation medium 4100 such as Optisol. In one embodiment, the preservation medium 4100 is withdrawn by the syringe 4000 from a storage container (not shown) containing the donor eye tissue. A filter 4200 may be placed on the end of the syringe 4000 and about 4 drops to about 5 drops of preservation medium 4100 may be pushed out of the syringe 4000 and onto a segment of sterile gauze by depressing a plunger 4200 of the syringe 4000.

The filter may then be coupled to the inlet port 3420 coupled to the second stopcock. The second stopcock is placed in an open position to allow the preservation medium 4100 in the syringe 4000 to be pushed into and through the second stopcock and into the second tube 3500. The plunger 4200 of the syringe 4000 is depressed until the preservation medium 4100 stored therein forces all of the fluid 3002 disposed downstream from the second stopcock to exit the second tube 3500 and AAC tube 2300 through exit aperture 2400 and preservation medium 4100 to emerge from the exit aperture 2400. In one embodiment, the plunger 4200 is depressed until a meniscus (not shown) of preservation medium 4100 forms along the top surface 2240 of the tissue pedestal 2200 of the AAC 2000. Then, the second stopcock is placed in a closed position and the syringe 4000 may be decoupled from the inlet port 3420 of the stopcock assembly 3400.

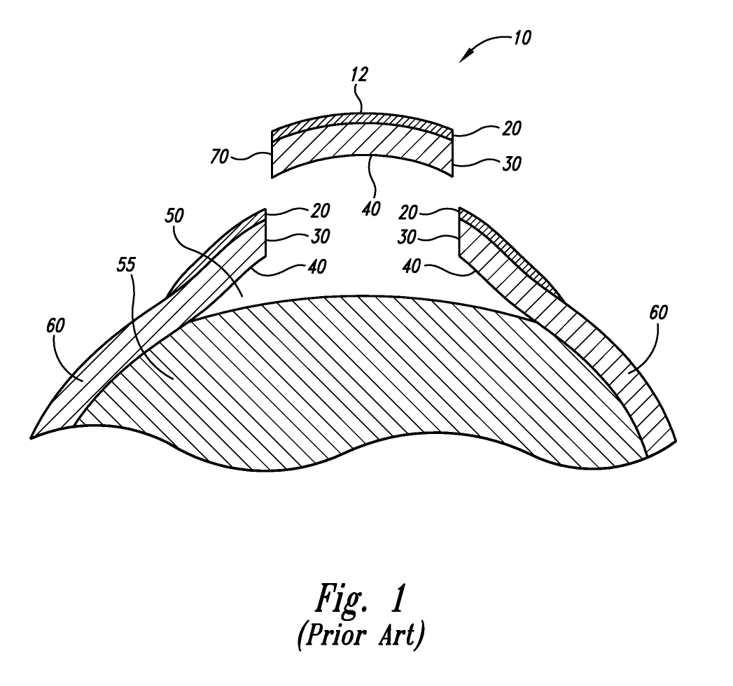

Returning to FIG. 4, in a next block 1270, the donor eye tissue may be mounted to the AAC 2000. Referring to FIG. 6C, a pair of forceps (not shown) may be used to remove the donor eye tissue from its storage container and place the donor eye tissue 10 and 60 on the top surface 2240 of the tissue pedestal 2200. Care may be taken during the mounting of the donor eye tissue to the AAC 2000 to avoid traumatizing the donor eye tissue (which includes the cornea 10 and sclera 60). In particular, it may be desirable to avoid trauma to the corneal endothelium 40 (see FIG. 1) of the cornea 10 through manipulation with the forceps and/or contact with the top surface 2240 of the tissue pedestal 2200.

In one embodiment, the donor eye tissue is centered on the top surface 2240 of the tissue pedestal 2200 with the corneal tissue disposed within the depression 2260. The collar 2500 may receive the tissue pedestal 2200 with the edges of the donor eye tissue along its perimeter sandwiched within the gap 2600 formed between the side surface 2220 of the tissue pedestal 2200 and the inside surface 2520 of the collar 2500. The edges of the donor eye tissue along its perimeter include a portion of the sclera 60. The collar 2500 clamps the portion of the sclera 60 along the perimeter of the donor eye tissue to the tissue pedestal 2200. An artificial anterior chamber 5000 may be defined between the cornea 10 and the tissue pedestal 2200 by a fluid tight seal formed between the sclera 60 and the side surface 2220 of the tissue pedestal 2200.

The AAC 2000 and mounted donor eye tissue may be placed under the head 1610 of the laser apparatus 1600. In one embodiment, the rigid pad 1800 is positioned under the head 1610 of the laser apparatus 1600. The pad 1800 may include a recess 1810 sized and shaped to receive the base 2100 of the AAC 2000. The recess 1810 may include a vertical sidewall 1812 that traverses the perimeter of the recess 1810 and extends upwardly from a bottom surface 1814 of the recess 1810. The base 2100 of the AAC 2000 may be inserted into the recess 1810 with the tissue pedestal 2200 extending upwardly above the recess 1810. The sidewall 1812 of the recess 1810 may prevent the base 2100 of the AAC 2000 from sliding on the bottom surface 1814 during processing. In embodiments wherein the sterile drape is disposed along the top surface 1802 of the rigid pad 1800, a hole may be cut in the sterile drape in the portion of the sterile drape adjacent to the recess 1810 to allow the base 2100 of the AAC 2000 to be received into the recess 1810.

Referring back to FIG. 4, in a next block 1280, the first pressure is used to fill the artificial anterior chamber 5000 with a fluid 4300. The fluid 4300 (which may include the fluid 3002 and/or preservation medium 4100) is disposed within the AAC tube 2300 and second tube 3500. Referring to FIG. 6C, the first stopcock of the stopcock assembly 3400 is placed in an open position to allow the fluid 3002 from the fluid source 3000 to flow into the first tube segment 3010, through the first stopcock. When the fluid 3002 from the fluid source 3000 engages the fluid 4300, the fluid 3002 exerts a pressure on the fluid 4300 that pushes or forces a portion of the fluid 4300 through both the second tube 3500 and AAC tube 2300 and out the exit aperture 2400. The force of the fluid 3002 acting upon the fluid 4300 forces a portion of the fluid 4300 out of the exit aperture 2400 disposed underneath a portion of the donor eye tissue. Because a fluid tight seal is formed along the perimeter of the donor eye tissue between the sclera 60 of the donor eye tissue and the tissue pedestal 2200, the fluid 4300 is trapped within the artificial anterior chamber 5000 formed behind the cornea 10. The pressure inside the artificial anterior chamber 5000 may be determined by the force exerted by the fluid 3002 on the fluid 4300.

Depending on the type of cornea transplant for which the corneal tissue was requested, the epithelium 20 (see FIG. 1) of the cornea 10 may be either removed or maintained intact. One of the technicians may remove the epithelium 20 by scraping it from the stroma 30 with a sterile cotton tip applicator or surgical spear. Any debris remaining on the stroma 30 may be rinsed away with a sterile ophthalmic rinse, preservation medium, or other suitable rinsing solution known in the art.

Referring back to FIG. 4, in a next block 1290, the amount of pressure in the artificial anterior chamber 5000 behind the cornea 10 of the donor eye tissue may be increased to a second pressure level. In one embodiment, the pressure is increased by raising the fluid source 3000, such as the IV bag 3004, to a second vertical height located above the first vertical height.

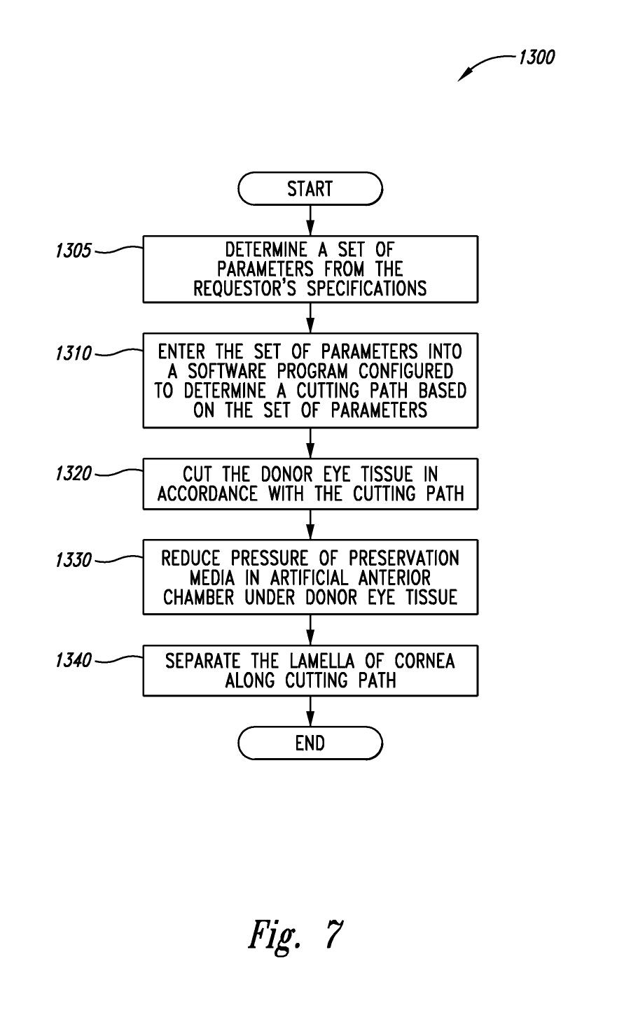

Referring back to FIG. 3, in a block 1300, the corneal tissue is removed from the donor eye tissue. The laser apparatus 1600 may be used to separate and/or cut the requested corneal tissue from the donor eye tissue using any method known in the art. In one non-limiting embodiment depicted in FIG. 7, the process of removing the corneal tissue from the donor eye tissue begins with a block 1310.

In a block 1305, a set of parameters is determined from the requestor's specifications. The set of parameters are used to communicate the requestor's specifications, such as the boundary of the corneal tissue or the location of incisions along the boundary, to a software program that designs a cutting path to be followed by a laser of the laser apparatus 1600. In some embodiments, the specifications provided by the requestor are used to derive or calculate the set of parameters. In other embodiments, the requestor's specifications include the set of parameters. In some embodiments, modifications to the specifications and/or set of parameters may be required. If modifications are required, the requestor may be consulted and/or may approve the modifications. Optionally, a pachymeter (not shown) may be used to measure the thickness of the cornea 10 near its center and along its periphery. The thickness measurements taken while the donor eye tissue is mounted to the AAC 2000 may be used to determine the set of parameters and/or suitability of the donor eye tissue for cutting in accordance with the requestor's specifications.

In some embodiments, the head 16 portion of the laser apparatus 1600 may be lowered and the tissue interface 1620 may physically contact the top surface of the cornea 10. The tissue interface 1620 may include a contact lens that applanates a portion of the cornea 10 of the donor eye tissue.

In a block 1310, the set of parameters are entered into the software program. The software program that designs the cutting path may be installed in a memory and executed by a processor incorporated into the laser apparatus 1600. The laser apparatus 1600 may include a user interface 1630 for communicating with the technician(s) and/or circulator(s). The technician(s) and/or circulator(s) may enter the set of parameters into the memory of the laser apparatus 1600 using the user interface 1630, which may include a standard keyboard 1632. The software program may provide information to the technician(s) and/or circulator(s) via the user interface 1630, which may include a standard computer display 1634. The computer display 1634 may be used to verify and/or modify the set of parameters entered into the memory using the keyboard 1632.

The software program uses the set of parameters to design the cutting path, which may include a photodisruption pattern. Because methods of using the set of parameters to design the cutting path and direct the laser of the laser apparatus 1600 to cut the donor eye tissue in accordance with the cutting path are well known in the art, they are not discussed in detail herein.

After the software has designed the cutting path, the method moves to a block 1320 wherein the donor eye tissue is cut according to the cutting path. In one embodiment, the software program directs the laser of the laser apparatus 1600 to cut the donor eye tissue without any human intervention in a completely automated process. Because the patient's cornea may be cut using the same or an identical laser apparatus 1600, executing the same or identical software program, and supplied with the same specifications, the piece of corneal tissue cut from the donor eye tissue may be substantially identical to the piece of corneal tissue removed from the patient's eye.

An example of a software program configured to design the cutting path with photodisruption incisions forming a photodisruption pattern, and direct the laser of the laser apparatus 1600 to cut the photodisruption incisions includes IntraLase-Enabled Keratoplasty™ (IEK) developed by Intralase Corp. for use with its INTRALASE® FS laser. The IEK software program includes a user interface into which the set of parameters specifying how to cut the donor eye tissue may be entered. The IEK software program then designs the cutting path that includes the photodisruption incisions. The IEK software program then directs the laser of the laser apparatus 1600 to cut the donor eye tissue in accordance with the cutting path.

Examples of parameter values included in the set of parameters that the IEK software program may utilize to design the cutting path and direct the laser to cut the donor eye tissue in accordance with the cutting path include parameters related to a lamellar cut, anterior side cut, and posterior side cut. The following table includes a list of example parameter values for each of these cuts.

| | Anterior | | |
|---|---|---|---|
| Lamellar Cut | Side Cut | Posterior Side Cut | Orientation Marks |
| Depth in cornea | Posterior depth | Anterior depth | Depth in cornea |
| Outer diameter | Diameter | Posterior depth | Width |
| Inner diameter | Energy | Diameter | Length |
| Energy | Cut position 1 | Energy | |
| | Cut angle 1 | Side cut angle | |
| | Side cut angle | | |

After the laser apparatus 1600 has finished cutting the donor eye tissue, the method progresses to a block 1330 wherein the pressure of the fluid 4300 in the artificial anterior chamber 5000 behind the cornea 10 is reduced. Reducing this pressure help prevent damage to the corneal tissue. In particular, it may be desirable to limit the duration of exposure of the corneal endothelium 40 (see FIG. 1) to high pressure. In one embodiment, the pressure is reduced by lowering the fluid source 3000 back to the first vertical position. Optionally, the surface of the cornea 10 may be marked in any manner known in the art including using a pen, marker, and the like.

In some embodiments, the contact lens of the tissue interface 1620 may be de-applanated from the donor eye tissue 10 and 60 and the tissue interface 1620 removed from the head 1610 of the laser. The tissue interface 1620 may be sterilized for reuse or discarded. In some embodiments, the tissue interface 1620 is disposable and discarded after each use. Optionally, the portion of the corneal tissue along the cutting path may be inspected visually using a optical device 1640 of the laser apparatus 1600, any suitable microscope (not shown), and the like.

After the cutting process of the laser apparatus 1600 has finished, the requested corneal tissue must be separated from the unwanted tissue. In a block 1340, the lamella of the cornea 10 are separated along the cutting path. This process is well known in the art and may be performed by an automated process. In one embodiment, a LASIK spatula or similar tool is used to loosen stromal adhesions and separate the lamella along the edges of the incisions made along the cutting path. Care may be taken to ensure detachment occurs along proper lamella and in the location of incisions such as laser photodisruption incisions.

Returning to FIG. 3, the method progresses to a block 1400. In the block 1400, the requested corneal tissue is packaged for shipment to the requestor and shipped to the requestor for transplantation into the patient. Packaging the corneal tissue may be accomplished using any method suitable for packaging donor eye tissue including placing the corneal tissue in a container (not shown), such as a glass jar with a lid, containing a preservative medium such as Optisol. In one embodiment, the corneal tissue is placed back in the storage container or vial previously used to store the donor eye tissue. The remaining portion of the donor eye tissue may be disposed of using any method known in the art. The corneal tissue may then be shipped to the requestor using any method known in the art for shipping donor eye tissue to the requestor of such tissue.

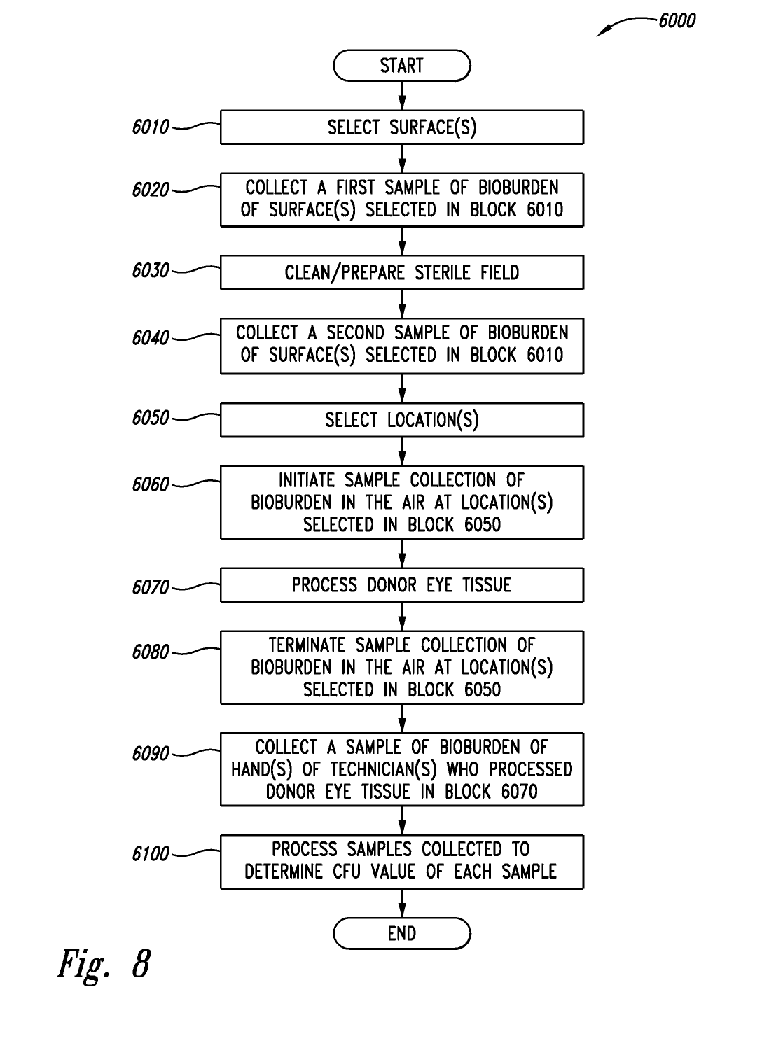

Turning now to FIG. 8, one aspect of the present invention includes a method 6000 for determining the bioburden of a laboratory (i.e., the number of microorganisms with which the laboratory is contaminated) before, during, and/or after processing donor eye tissue. As used herein, the term “microorganisms” refers to bacterium and fungi. The method 6000 may be used to determine whether the bioburden of the laboratory is small enough to permit the laboratory to process donor eye tissue for the purposes of providing corneal tissue to the requestor. For example, the method 6000 may be used to determine whether the laboratory may safely process donor eye tissue in accordance with the method 1000 described above. In some embodiments, the method 6000 may be used to determine whether the laboratory that does not qualify as a clean room may safely process donor eye tissue for transplantation into patients.

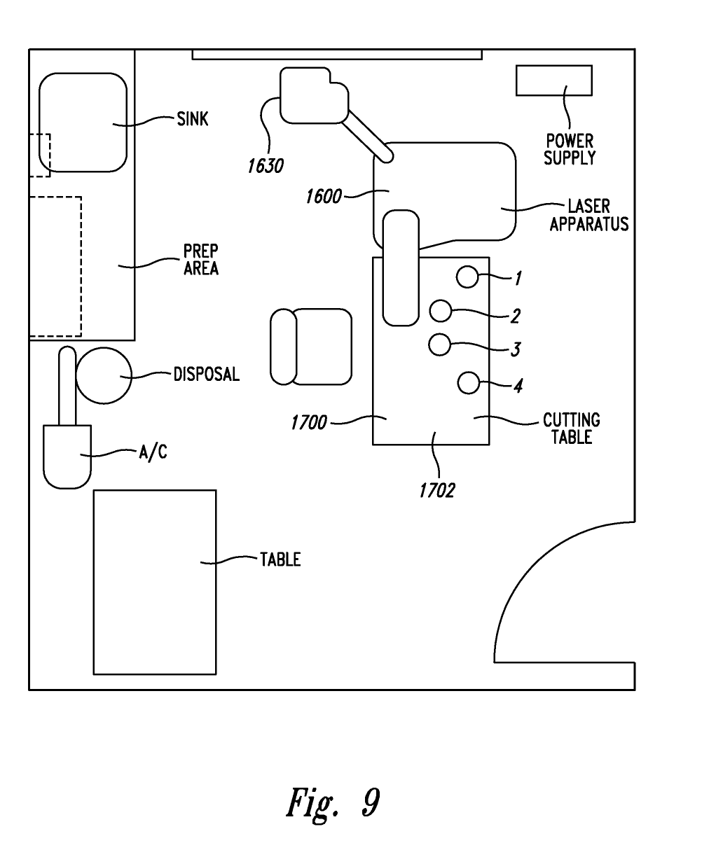

The method 6000 starts with a block 6010 wherein a surface is selected. The surface may include any surface in the laboratory including any surface upon which donor eye tissue is processed, such as the work surface 1702. In one embodiment, the work surface 1702 is selected. As is appreciated by those of ordinary skill in the art, multiple samples of the same surface and/or samples of more than one surface may be collected. For example, referring to FIG. 9, locations 2 and 3 may be suitable locations on the work surface 1702 for collecting samples of the bio-burden of the work surface 1702.

After the surface is selected, in a block 6020, a first sample of the bioburden of the surface is collected. The first sample may be collected by exposing a material upon which microorganisms may live, grow, and/or form colonies (referred to hereafter as “growth medium”), such as an agar containing nutrients, to the surface. As is appreciated by those of ordinary skill in the art, the growth medium may include compounds that promote the growth of certain microorganisms and prevent or limit the growth of other microorganisms, and growth medium including such compounds are within the scope of the present invention.

In one embodiment, the growth medium is housed within a container, such as a Petri dish and the like, with a lid. One non-limiting example of a suitable growth medium for use with surface testing includes a letheen agar. A suitable letheen agar disposed within a Petri dish may be purchased under the tradename “Replicate Organism Direct Agar Contact plates” or RODAC® plates from Laboratories at Bonfils (also known as “LABS”) of 717 Yosemite Street, Denver, Colo. 80230.

The first sample may be collected by removing the lid from the container and placing the top surface of the growth medium in direct contact with the surface selected in the block 6010. It may be beneficial to place the entire surface of the growth medium in contact with the selected surface. Pressure may be applied to the underside of the container and/or the container may be rocked back and forth to ensure the entire surface of the growth medium or a substantial portion thereof contacts the selected surface. To avoid contaminating or otherwise disturbing the top surface of the growth medium, care should be taken to avoid touching the growth medium.

Contact between the surface of the growth medium and the selected surface may be maintained for a predetermined period of time. In one embodiment, contact between the surface of the growth medium and the selected surface is maintained for about 3 seconds to about 5 seconds.

After the predetermined period of time has elapsed, contact between the growth medium and the selected surface may be discontinued. The lid may be placed on the container. Optionally, the bottom of the container may be labeled with information related to the sample collected. The information may include the date the first sample was collected, identification of the selected surface, location of the selected surface, identification of the technician collecting the first sample, a time the first sample was collected, the period of time the growth medium inside the container was in direct contact with the selected surface, and any other information related to the sample collected and/or laboratory.

Optionally, the lid of the container housing the growth medium may be secured to the container to prevent subsequent exposure of the growth medium to the environment outside the container. The lid may be secured to the container using any method known in the art, including taping the lid to the container using any suitable tape known in the art. The container may be stored with the surface of the growth medium that was placed in contact with the selected surface facing upward.

Optionally, information related to the first sample may be recorded. The information recorded may include the same information used to label the first sample.

In a block 6030, the selected surface and/or sterile field is cleaned and/or prepared by the technician. In one embodiment, the process of the block 6030 may be conducted in accordance with the process of the block 1220 (see FIG. 4). The process of cleaning and/or preparing the selected surface and/or sterile field is well known in the art and the invention is not limited by the process used to clean the selected surface and/or prepare the sterile field.

In a block 6040, a second sample of the bioburden of the surface is collected. The second sample may be collected in the same manner the first sample was collected. Further, the second sample may be labeled, recorded, and stored in the same manner described above with respect to the first sample.

The processes of the blocks 6010 through 6040 may be used to collect samples to assess the procedure of cleaning/preparing the selected surface/sterile field, aseptic technique of the technicians performing the procedure, and/or the ability of the technician to perform the procedure.

In blocks 6050 through 6080, samples of the bio-burden of the air in which the donor eye tissue and/or corneal tissue is processed are collected. Exposure of a top surface of a growth medium to the air and allowing microorganisms to settle thereupon may be an appropriate, easy, and inexpensive method of obtaining a representation of the bioburden settling from the air at the location of the top surface of the growth medium.

In a block 6050, one or more locations at which samples will be collected are selected. The locations may include any location in the laboratory including locations on any work surface upon which donor eye tissue is processed, locations at or near the edge of the sterile field, and the like. In one embodiment, one or more location along the edge of the sterile drape are selected. Referring to FIG. 9, locations 1 and 4 may be suitable locations for collecting samples of the bio-burden of the air of the laboratory.

After the location(s) is/are selected, in a block 6060, sample collection of the bioburden of the air at the selected location(s) is initiated. Sample collection may be initiated by exposing a growth medium to the air at the location(s). In one non-limiting example, the growth medium used to sample the air at the selected location(s) includes trypticase soy agar (“TSA”). The TSA may be housed in a container with a lid such as a Petri dish. The Petri dish may have a diameter of about 100 mm. Sample collection may be initiated by removing the lid of a single container (e.g., the Petri dish) containing growth medium (e.g., the TSA) to expose the top surface of the growth medium to the air at each of the selected location(s). To avoid contaminating or otherwise disturbing the growth medium, care should be taken to avoid touching the growth medium. The top surface of the growth medium may be exposed to the air from the beginning to the end of the processing of the donor eye tissue and/or for a predetermined period of time.

In an alternate embodiment, sample collection of the bioburden of the air at the selected location(s) may be initiated after the laboratory has started processing the donor eye tissue or at other times during the performance of the method 6000.

In a block 6070, the donor eye tissue is processed by the laboratory. Processing the donor eye tissue exposes the donor eye tissue to the air in the laboratory, which may contaminate or be contaminated by the donor eye tissue. Contamination in the air may subsequently settle on technicians and/or surfaces present in the laboratory, such as the surface of the growth medium exposed to the air at the selected location. In one embodiment, the process performed in the block 6060 may be substantially similar to the processes performed in the blocks 1200 to 1400 of FIG. 3 described above.

In some embodiments, a first sterile transport swab is used to swab the donor eye tissue immediately after it is exposed to the air in the laboratory and a second sterile transport swab is used to swab the donor eye tissue immediately before it is packaged and thereby isolated from the air in the laboratory. In some embodiments, the first and second sterile transport swabs are dipped in saline before swabbing the donor eye tissue.

Any microorganisms transferred to the first sterile transport swab by swabbing the donor eye tissue may be transferred to the top surface of a growth medium by swabbing the first sterile transport swab on the top surface of the growth medium. Any microorganisms transferred to the second sterile transport swab may be transferred to the top surface of another growth medium. A suitable growth medium includes any growth medium suitable for collecting samples of the selected surface in the blocks 6020 and 6040. The growth medium may be housed in any container suitable for housing the growth medium used in the blocks 6020 and 6040.

Each of the containers may be labeled with information related to the sample collected therein. The information may include the date the sample was collected, identification of the donor eye tissue, identification of the technician collecting the sample, a time the sample was collected, and any other information related to the sample collected and/or laboratory. Optionally, information related to each of the samples may be recorded. The information recorded may include the same information used to label the containers.

In an alternate embodiment, the microorganism transferred to the first and second sterile transport swabs are not transferred to the growth media. Instead, the first and second sterile transport swabs are packaged separately for further processing by an offsite laboratory. Each of the packages may be labeled with information related to the sample collected therein. The information may include the date the sample was collected, identification of the donor eye tissue, identification of the technician collecting the sample, a time the sample was collected, and any other information related to the sample collected and/or laboratory. Optionally, information related to each of the samples may be recorded. The information recorded may include the same information used to label the packages. The offsite laboratory may transfer the microorganisms on each of the sterile transport swabs onto the surface of a growth medium as described above.

The samples collected from the surface of the donor eye tissue may be used to determine the bioburden of the tissue before and after exposure to the environment in the laboratory.

When donor eye tissue processing is completed or the predetermined period of time has elapsed, in a block 6080, sample collection of the bioburden of the air is terminated. In an alternate embodiment, sample collection of the bioburden of the air at the selected location(s) may be terminated before the laboratory has completed processing the donor eye tissue or at other times during the performance of the method 6000. Sample collection may be terminated by placing the lid(s) on the container(s) housing the growth medium.

Optionally, each of the containers may be labeled with information related to the sample collected therein. The information may include the date the sample was collected, identification of the selected location, identification of the technician collecting the sample, a time the sample was collected, the period of time the growth medium inside the container was exposed to the air at the selected location and any other information related to the sample collected and/or laboratory.

Optionally, the lid(s) of the container(s) housing the growth medium may be secured to the container(s) to prevent subsequent exposure of the growth medium to the environment outside the container. The lid(s) may be secured to the container(s) using any method known in the art, including taping each of the lids to its corresponding container using any suitable tape known in the art. The container(s) may be stored with the surface of the growth medium that was exposed to the air at the selected location facing upward.

Optionally, information related to each of the samples may be recorded. The information recorded may include the same information used to label the container(s).

In a block 6090, a sample of the bioburden present on the surface of the hand(s) of the technician(s) who processed the donor eye tissue in the block 6070 is collected. A container with a lid housing a growth medium substantially similar to the container and growth medium used to collect the first and second samples of the selected surface in the blocks 6020 and 6040 may be used to collect the sample from the hand(s) of the technician(s). Because the technician(s) may be wearing gloves while processing the donor eye tissue, it may be desirable to sample the outside surface of the gloves including the portion of the outside surface of the gloves of each technician adjacent to the technician's fingers.

In one embodiment, each technician who processed the donor eye tissue in the block 6070 collects the sample of the bioburden present on the surface of his/her hand(s). First, the portion of the outside surface of each glove of the technician to be sampled is identified. In one embodiment, the portions of the glove adjacent to the fingertips of the dominant hand of the technician are to be sampled. To collect the sample, the technician removes the lid from the container taking care not to touch the growth medium with portions of the outside surface of his/her gloves other than those identified. The bottom of the container may be held between the thumb and the index and second fingers of the non-dominant hand. The portions of the glove adjacent to the fingertips of the dominant hand of the technician may be sampled by pressing the gloved fingertips of the dominant hand against the surface of the growth medium. Transference from the surface of the glove to the growth medium may be increased by rolling or rocking the gloved fingertips against the surface of the growth medium.

After sampling is complete, the gloves may be cleaned and/or sanitized or discarded. In one embodiment, the gloves are cleaned with a solution such as 70% isopropyl alcohol and the like.

Optionally, each of the containers may be labeled with information related to the sample collected therein. The information may include the date the sample was collected, identification of the technician whose hand(s) were sampled, identification of the technician collecting the sample, a time the sample was collected, and any other information related to the sample collected and/or laboratory.

Optionally, the lid(s) of the container(s) housing the growth medium may be secured to the container(s) to prevent subsequent exposure of the growth medium to the environment outside the container. The lid(s) may be secured to the container(s) using any method known in the art, including taping each of the lids to its corresponding container using any suitable tape known in the art. The container(s) may be stored with the surface of the growth medium that was touched by the technician facing upward.

Optionally, information related to each of the samples may be recorded. The information recorded may include the same information used to label the container(s).

In a block 6100, the samples collected are processed to determine a colony-forming unit (“CFU”) value of each sample. The samples collected in the blocks 6020 and 6040 may be processed to determine the CFU value of the surface(s) selected in the block 6010. The samples collected in the blocks 6060 through 6080 may be processed to determine the CFU value of the air at the location(s) selected in the block 6050. The samples collected using the first and second transport swab in the block 6070 may be processed to determine the CFU value of the surface of the donor eye tissue. The samples collected in the block 6090 may be processed to determine the CFU value of the surface of the hand(s) of the technician(s) who processed donor eye tissue in the block 6070

Individual microorganisms on the surface of the growth medium may grow separate and distinguishable colonies. The number of colonies may be counted to provide a CFU value for each sample collected. Because dead microorganisms do not form colonies, the CFU value is reflective of the number of living microorganisms present in the sample before the sample was incubated. The number of living organisms in each sample may be reflective of the bioburden of the surface(s) selected in the block 6010, the air at the location(s) selected in the block 6050, and the surface of the hand(s) of the technician(s) who processed donor eye tissue in the block 6070.

The CFU value present in each of the samples collected is determined by incubating each sample to encourage any microorganisms on the surface of the growth medium to grow and create colonies. The incubation process may include a first aerobic incubation at a temperature of about 30° C. to about 35° C. for about two days to about four days, followed by a second aerobic incubation at a temperature of about 20° C. to about 25° C. for about three days to about five days. The total duration of incubation may be about seven days.

After the incubation process is complete, the number of microbial colonies visible to the unaided eye is counted. Colonies that extend outwardly to form a large colony are counted as a single colony no matter how much area is occupied by the colony. Each colony may be gram stained and its macroscopic colonial morphology observed. Optionally, the species of the microorganisms living within the colony may be identified.

In some embodiments, the incubation process of the block 6100 includes sending or shipping the samples collected in the blocks 6020, 6040, 6060-6080, and 6090 to a reference laboratory for incubation. A non-limiting example of a laboratory suitable for performing the incubation process includes the Laboratories at Bonfils mentioned above. The Laboratories at Bonfils may also process the packaged sterile transport swabs including samples collected from the surface of the donor eye tissue to obtain the CFU value of the swabs. Optionally, the reference laboratory may count the colony forming units and/or identify the microorganisms living within one or more of the colonies.

If one or more of the samples collected is to be shipped to the reference laboratory within about 24 hours, the sample(s) collected to be shipped may be stored at room temperature prior to shipment. It may be desirable to avoid exposing the sample(s) collected to temperature extremes. In one embodiment, the sample(s) collected is/are stored at a temperature between about 32° F. and about 120° F. If the sample(s) collected is/are not shipped within 24 hours, the sample(s) collected may be refrigerated. In one embodiment, the sample(s) collected may be refrigerated for up to about 48 hours before shipment.

The CFU values of the samples may be compared to detect changes in the number of microorganisms present. For example, the CFU value of the sample collected from the surface selected in the block 6010 before cleaning the selected surface may be compared to the CFU value of the sample collected from the selected surface after cleaning the selected surface. Similarly, the CFU value of the sample collected from the surface of the donor eye tissue before the corneal tissue is extracted from the donor eye tissue may be compared to the CFU value of the sample collected from the surface of the donor eye tissue after the corneal tissue is extracted from the donor eye tissue.

The method 6000 may be repeated and the samples may be collected in an identical manner each time the method is repeated. In this manner, each sample collected during a single repetition corresponds to a sample collected in each of the other repetitions. In one embodiment, the method 6000 is repeated ten times. The CFU value for each sample and its corresponding samples may be determined. The CFU value for a particular sample and its corresponding samples may be used to establish statistics for samples collected in the same manner as the particular sample. The statistics may include the mean and standard deviation of the CFU value for samples collected in the same manner as the particular sample. For example, the mean and standard deviation may be found over a predetermined number of repetitions of the method 6000 for the CFU value of the sample collected in the block 6040 from the work surface 1702 after the surface has been cleaned.

The mean and standard deviation for a particular sample and its corresponding samples may be determined and used to determine whether the bioburden of a next sample collected in the same manner as the particular sample has become too great. In one embodiment, the mean and standard deviation are used to determine a threshold value that indicates whether the bioburden of the next sample collected is too great. For example, the threshold value may be set equal to the sum of the mean and two standard deviations or the sum of the mean and three standard deviations. If the bioburden of a next sample collected has become too great, special cleaning procedures may be implemented or the processing of human tissue may be terminated.

The method 6000 may be used to determine whether the laboratory has become contaminated by bio-contamination, and/or the effectiveness of the bio-contamination control procedures of the laboratory. Additionally, the method 6000 may be used to assess the cleaning procedures, aseptic technique of the technicians performing the processing, and the viable air bio-burden that the donor eye tissue and/or corneal tissue was exposed to during processing. The method 6000 may demonstrate the effect of the presence and movement of the technician(s) and the bioburden present in the surrounding environment.

As part of an approval or validation process, before the laboratory may initiate processing donor eye tissue for transplantation into patients, the method 6000 may be repeated ten times. Tissues processed with during the ten repetition of method 6000 may be unsuitable for corneal transplant into a patient. The CFU values obtained for each sample during each repetition of the method 6000 may be used to determine the mean and standard deviation for each type of sample collected.

If a laboratory is processing donor eye tissue using the method 1000 or another method, it may be beneficial to perform the method 6000 periodically to determine whether the laboratory has become contaminated through processing donor eye tissue. For example, the method 6000 may be performed once a quarter or every six months to determine whether the laboratory has an increased bio-burden level.

If the growth medium used in any of the blocks of method 6000 is stored at a temperature below the temperature of the laboratory, it may be desirable to place the growth medium in a location having a temperature substantially similar to the temperature of the laboratory before initiating sample collection. In one embodiment, the growth medium is placed in a location having a temperature substantially similar to the temperature of the laboratory at least 30 minutes prior to initiating sample collection to allow the growth medium to warm to approximately the temperature of the laboratory.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. A method of preparing custom corneal tissue in response to a request from a requestor wherein the request comprises specifications specifying the size and shape of the custom corneal tissue, the method comprising:
receiving the request for the custom corneal tissue;
selecting donor eye tissue from a plurality of donor eye tissues;
preparing the selected donor eye tissue to be cut according to the specifications of the request;
determining a set of parameters from the specifications;
designing a cutting path based on the set of parameters;
cutting the selected donor eye tissue along the cutting path to produce the custom corneal tissue;
packaging the custom corneal tissue; and
providing the packaged custom corneal tissue to the requestor.

2. The method of claim 1, wherein the plurality of donor eye tissues are stored in an eye bank facility.

3. The method of claim 1, wherein providing the packaged custom corneal tissue to the requestor comprises shipping the packaged custom corneal tissue to the requestor.

4. The method of claim 1, wherein designing the cutting path based on the set of parameters comprises entering the set of parameters into a software program configured to receive the set of parameters and design the cutting path based on the set of parameters.

5. The method of claim 1, wherein cutting the custom corneal tissue from the selected donor eye tissue along the cutting path comprises cutting the donor eye tissue with a laser apparatus configured to follow the cutting path.

6. The method of claim 1, wherein the cutting path comprises a plurality of incisions and cutting the selected donor eye tissue along the cutting path comprises making each of the incisions of the plurality in the selected donor eye tissue.

7. The method of claim 6, wherein each of the incisions of the plurality of incisions comprise a photodisruption pattern and making each of the incisions of the plurality in the selected donor eye tissue comprises directing the laser apparatus to create the photodisruption pattern of each of the incisions of the plurality of incisions in the selected donor eye tissue.

8. The method of claim 1, wherein the cutting path comprises a photodisruption pattern.

9. The method of claim 1, further comprising separating the custom corneal tissue from the selected donor eye tissue after cutting the selected donor eye tissue along the cutting path.

10. The method of claim 1, wherein preparing the selected donor eye tissue to be cut according to the specifications of the request comprises mounting the donor eye tissue to an artificial anterior chamber assembly and creating an artificial anterior chamber between the donor eye tissue and the artificial anterior chamber assembly.

11. The method of claim 10, wherein the artificial anterior chamber is filled with a fluid having a selected pressure, the method further comprising reducing the selected pressure of the fluid in the artificial anterior chamber after cutting the selected donor eye tissue along the cutting path.

12. The method of claim 10, wherein the artificial anterior chamber is filled with a fluid having a selected initial pressure and preparing the donor eye tissue to be cut according to the specifications of the request comprises increasing the pressure of the fluid in the artificial anterior chamber above the selected initial pressure prior to cutting the selected donor eye tissue along the cutting path.

13. The method of claim 12, comprising reducing the pressure of the fluid in the artificial anterior chamber below the increased pressure used for cutting after cutting the selected donor eye tissue along the cutting path.

14. A method of providing a replacement corneal tissue configured to replace a substantially identical corneal tissue in a patient, the method comprising:
receiving specifications from a requestor describing the replacement corneal tissue;
using the specifications to determine a set of parameters describing how to cut the donor eye tissue;
selecting a donor eye tissue from a plurality of donor eye tissues;
using the set of parameters to determine an outer boundary of the replacement corneal tissue;
using the outer boundary of the replacement corneal tissue to design a cutting path;
cutting the selected donor eye tissue along the cutting path to produce the replacement corneal tissue;
packaging the replacement corneal tissue; and
providing the packaged replacement corneal tissue to the requestor.

15. The method of claim 14, wherein the plurality of donor eye tissues are stored in an eye bank facility.

16. The method of claim 14, wherein using the set of parameters to determine the outer boundary of the replacement corneal tissue comprises entering the set of parameters into a software program configured to receive the set of parameters and determine the outer boundary of the replacement corneal tissue using the set of parameters.

17. The method of claim 14, wherein the selected donor eye tissue along the cutting path comprises cutting the selected donor eye tissue with a laser apparatus configured to follow the cutting path.

18. The method of claim 14, wherein the cutting path comprises a photodisruption pattern.

19. The method of claim 14, wherein the cutting path comprises a plurality of incisions and cutting the selected donor eye tissue along the cutting path comprises making each of the incisions of the plurality in the selected donor eye tissue.

20. The method of claim 19, wherein each of the incisions of the plurality of incisions comprise a photodisruption pattern and making each of the incisions of the plurality in the selected donor eye tissue comprises directing the laser apparatus to create the photodisruption pattern of each of the incisions of the plurality of incisions in the selected donor eye tissue.

21. The method of claim 14, further comprising separating the custom corneal tissue from the selected donor eye tissue after cutting the selected donor eye tissue along the cutting path.

22. The method of claim 14, further comprising mounting the selected donor eye tissue to an artificial anterior chamber assembly and creating an artificial anterior chamber between the donor eye tissue and the artificial anterior chamber assembly.

23. The method of claim 22, wherein the artificial anterior chamber is filled with a fluid having a selected pressure, the method further comprising reducing the selected pressure of the fluid in the artificial anterior chamber after cutting the selected donor eye tissue along the cutting path.

24. The method of claim 22, wherein the artificial anterior chamber is filled with a fluid having a selected initial pressure, the method further comprising increasing the pressure of the fluid in the artificial anterior chamber above the selected initial pressure prior to cutting the selected donor eye tissue along the cutting path.

25. The method of claim 24, further comprising reducing the pressure of the fluid in the artificial anterior chamber below the increased pressure used for cutting after cutting the selected donor eye tissue along the cutting path.

26. A method of determining the bioburden of a laboratory extracting corneal tissue from donor eye tissue by a person using a hand wearing a glove, the method comprising:
selecting a surface in the laboratory;
collecting a first sample of a portion of microorganisms on the surface selected;
after collecting the first sample, cleaning the surface selected;
after cleaning the surface selected, collecting a second sample of a portion of microorganisms on the surface selected;
collecting a third sample of a portion of microorganisms in the air in the laboratory during the extraction of corneal tissue from donor eye tissue;
collecting a fourth sample of a portion of microorganisms on the surface of the glove on the hand of the person extracting corneal tissue from donor eye tissue;
incubating the first, second, third, and fourth samples to determine a colony forming units value of each sample; and
determining the bioburden of the laboratory using the colony forming units values.

27. The method of claim 26, further comprising terminating further extractions of corneal tissue from donor eye tissue by the laboratory if the determined bioburden is above a predetermined threshold value.

28. The method of claim 26, wherein determining the bioburden of the laboratory using the colony forming units values comprises comparing the colony forming units values corresponding to at least one of the first, second, third, and fourth samples to a predetermined threshold value.

29. The method of claim 26, further comprising determining an acceptable level of bioburden of the laboratory using the colony forming units values of at least one of the first, second, third, and fourth samples.

30. The method of claim 26, wherein collecting the third sample of the portion of microorganisms in the air in the laboratory during the extraction of corneal tissue from donor eye tissue comprises:
before extracting corneal tissue from donor eye tissue, initiating sample collection of the portion of microorganisms in the air in the laboratory;
extracting corneal tissue from donor eye tissue; and
after extracting corneal tissue from donor eye tissue, terminating sample collection of the portion of microorganisms in the air in the laboratory.

31. The method of claim 26, wherein determining the bioburden of the laboratory using the colony forming units values comprises comparing a bioburden of the surface of the donor eye tissue before extracting corneal tissue from donor eye tissue to a bioburden of the surface of the donor eye tissue after extracting corneal tissue from donor eye tissue, the method further comprising:
before extracting corneal tissue from donor eye tissue, collecting a fifth sample of a portion of microorganisms on the surface of the donor eye tissue;
after extracting corneal tissue from donor eye tissue, collecting a sixth sample of a portion of microorganisms on the surface of the donor eye tissue;
incubating the fifth and sixth samples to determine a colony forming units value of the fifth and sixth samples;
determining the bioburden the surface of the donor eye tissue before extracting corneal tissue from donor eye tissue using the colony forming units value of the fifth sample; and
determining the bioburden the surface of the donor eye tissue after extracting corneal tissue from donor eye tissue using the colony forming units value of the sixth sample.
